(19) United States
(12) Patent Application Publication
(10) Pub. No.: US 2005/0250941 A1
(43) Pub. Date: Nov. 10, 2005

(54) Synthesis of purified, moderately esterified polyol polyester fatty acid compositions

(75) Inventor: Jared John Schaefer, Wyoming, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/841,307

(22) Filed: May 7, 2004

(51) Int. Cl. C07H 13/02

(52) U.S. Cl. 536/119

(57) ABSTRACT

Processes for the preparation of a purified moderately esterified polyol fatty acid polyester composition including the steps of: (a) forming an initial reaction mixture, said initial reaction mixture containing: a polyol portion; a highly esterified polyol fatty acid polyester; a solvent; and, a catalyst, wherein the molar ratio of said polyol portion to said highly esterified polyol polyester is chosen such that the final ratio of total fatty acid esters to total polyol backbones is in the range from about 3.2:1 to about 6.4:1; wherein the molar ratio of said catalyst to said highly esterified polyol polyester is in the range of from about 0.01:1 to about 10:1, and wherein the weight ratio of said solvent to the combined weight of said polyol portion, said highly esterified polyol polyester, and said catalyst is in the range of from about 0.01:1 to about 2:1; and (b) forming an initial reaction product by reacting said initial reaction mixture in an inert atmosphere, in the presence of a sufficient amount of agitation, for a period of time in the range of from about 30 minutes to about 6 hours, and at a temperature in the range of from about 80° C. to about 140° C.

### CROSS-REFERENCE TO RELATED APPLICATIONS

Under 35 USC § 120, this application claims benefit of U.S. patent application Ser. No. 10/156,479, filed May 28, 2002; U.S. patent application Ser. No. 10/156,437, filed May 28, 2002; and U.S. patent application Ser. No. 10/156,476 filed May 28, 2002.

### BRIEF DESCRIPTION OF THE INVENTION

This invention relates to processes for the production of moderately esterified polyol fatty acid polyesters. More particularly, this invention relates to processes for preparing purified, moderately esterified polyol fatty acid polyesters that include aqueous and alcohol based purification processes, and the products made according to those processes.

### BACKGROUND OF THE INVENTION

As a result of their physical properties, moderately esterified polyol fatty acid polyesters are often used as surfactants or lubricants in various laundry, textile, lubricant and cosmetic compositions.

There are various techniques available for the synthesis of these moderately esterified polyol fatty acid polyesters. Although conventional processes for the manufacture of moderately esterified polyol fatty acid polyesters have known utilities, they often suffer from several deficiencies, most notable of which include poor reaction control and/or the need for expensive and/or complex purification techniques. In addition, these known processes are often unable to accurately predict and consistently control the exact composition of the finished product without the use of complex sampling and/or control modification procedures throughout the reaction process.

These known processes also often suffer from an inability to accurately control the average degree of esterification in the final moderately esterified polyol polyester compositions. Moreover, the moderately esterified polyol polyester compositions produced from these known synthesis techniques frequently contain unacceptable levels of impurities, such as solvent, polyol, lower alkyl esters, ash, soap, free fatty acids, and other unwanted reaction byproducts.

These limitations have heretofore constrained the industrial applicability and cost effective commercialization of these compounds in various laundry, textile, lubricant, and cosmetic applications requiring higher purity and/or greater predictability.

Accordingly, there is a need for processes for the synthesis of purified, moderately esterified polyol polyesters that allow for the production of polyol polyesters with the degree of purity necessary for widespread incorporation into a variety of industrial and commercial applications. There is also a need to provide purified, moderately esterified polyol polyester compositions with a degree of purity sufficient to be used in a variety of industrial and commercial applications. In addition, there is a need to provide processes for the production of purified polyol polyesters that are efficient, cost effective, and require less purification than those now known and employed in the art. Finally, there is also a need to provide processes that result in products with a degree of esterification that is highly controllable and/or reproducible.

### SUMMARY OF THE INVENTION

The present invention relates to processes for the production of purified, moderately esterified polyol fatty acid polyesters and the compositions made from those processes. More particularly, this invention relates to processes for preparing moderately esterified polyol fatty acid polyesters that include aqueous and alcohol based purification processes.

In one embodiment, the process for the preparation of a purified moderately esterified polyol fatty acid polyester composition includes the steps of:

a) forming an initial reaction mixture containing:
i) a polyol portion;
ii) a highly esterified polyol fatty acid polyester;
iii) a solvent; and,
iv) a catalyst,
   where the molar ratio of the polyol portion to the highly esterified polyol polyester is chosen such that the final ratio of total fatty acid esters to total polyol backbones is in the range from about 3.2:1 to about 6.4:1; where the molar ratio of the catalyst to highly esterified polyol polyester is in the range of from about 0.01:1 to about 10:1, and where the weight ratio of the solvent to the combined weight of the polyol portion, the highly esterified polyol polyester, and the catalyst is in the range of from about 0.01:1 to about 2:1;

b) forming an initial reaction product by reacting the initial reaction mixture in an inert atmosphere, in the presence of a sufficient amount of agitation, for a period of time in the range of from about 30 minutes to about 6 hours, and at a temperature in the range of from about 80° C. to about 140° C.;

In another embodiment, the present invention includes a process for the preparation of a purified moderately esterified polyol fatty acid polyester composition including the steps of:

a) forming an initial reaction mixture containing:
i) a polyol portion;
ii) a highly esterified polyol fatty acid polyester having a degree of esterification in excess of about 80%;
iii) a solvent; and,
iv) a catalyst,
   where the molar ratio of the polyol portion to the highly esterified polyol polyester is chosen such that the final ratio of total fatty acid esters to total polyol backbones is in the range of from about 3.2:1 to about 6.4:1; wherein the molar ratio of the catalyst to the highly esterified polyol polyester is in the range of from about 0.01:1 to about 10:1, and wherein the weight ratio of the solvent to the combined weight of the polyol portion, the highly esterified polyol polyester, and the catalyst is in the range of from about 0.01:1 to about 2:1;

b) forming an initial reaction product by reacting the initial reaction mixture in an inert atmosphere, in the presence of a sufficient amount of agitation, for a period of time in the range of from about 30 minutes to about 6 hours, and at a temperature in the range of from about 80° C. to about 140° C.;

c) adding an acid to the initial reaction product to neutralize any remaining catalyst, wherein the molar ratio of the acid to the catalyst is in the range of from about 0.01:1 to about 1:1;

d) forming a secondary reaction product by reacting the initial reaction product at a temperature-pressure combination at which distillation of the solvent occurs, wherein the pressure is in the range of from about 0.01 mmHg to about 760 mmHg, for a period of time in the range of from about 30 minutes to about 4 hours;

e) forming a purified reaction product by washing the secondary reaction product with a solvent free water washing solution, the solvent free water washing solution containing:
(i) from about 0% to about 5% of a salt; and
(ii) from about 95% to about 100% water;
   where the weight ratio of the water washing solution to the secondary reaction product is in the range of from about 0.01:1 to about 1:1, wherein the temperature of the secondary reaction product and the water wash solution are in the range of from about 20° C. to about 100° C., and where the wash time is in the range of from about 5 minutes to about 30 minutes;

f) isolating and removing impurities from the purified reaction product;

g) optionally repeating steps (e) and (f) for a number of times in the range of from about 1 to about 20; and, h) optionally drying the purification reaction product.

In another embodiment, the present invention relates to a process for the preparation of a purified moderately esterified sucrose fatty acid polyester composition, comprising the steps of:

a) forming an initial reaction mixture containing:
i) a sucrose polyol portion;
ii) a highly esterified sucrose fatty acid polyester having a degree of esterification in excess of 80%;
iii) a solvent selected from dimethyl formamide, dimethyl sulfoxide, acetonitrile, acetone, and mixtures thereof.; and,
iv) a catalyst,
   wherein the molar ratio of the polyol portion to the highly esterified polyol polyester is chosen such that the final ratio of total fatty acid esters to total polyol backbones is in the range from about 3.2:1 to about 6.4:1; wherein the molar ratio of the catalyst to the highly esterified polyol polyester is in the range of from about 0.01:1 to about 10:1, and wherein the weight ratio of the solvent to the combined weight of the polyol portion, the highly esterified polyol polyester, and the catalyst is in the range of from about 0.01:1 to about 2:1;

b) forming an initial reaction product by reacting the initial reaction mixture in an inert atmosphere selected from nitrogen, argon, helium, and combinations thereof, in the presence of a sufficient amount of agitation, for a period of time in the range of from about 30 minutes to about 6 hours, and at a temperature in the range of from about 80° C. to about 140° C.;

c) adding an acid to the initial reaction product to neutralize any remaining catalyst, wherein the molar ratio of the acid to the catalyst is in the range of from about 0.01:1 to about 1:1;

d) forming a secondary reaction product by reacting the initial reaction product at a temperature-pressure combination at which distillation of the solvent occurs, wherein the pressure is in the range of from about 0.01 mmHg to about 760 mmHg, for a period of time in the range of from about 30 minutes to about 4 hours;

e) forming a purified reaction product by washing the secondary reaction product with an alcohol washing solution, wherein the alcohol washing solution comprises an alcohol selected from alcohols with a carbon chain length in the range of from about 2 atoms to about 5 atoms, wherein the weight ratio of the alcohol washing solution to the secondary reaction product is in the range of from about 0.01:1 to about 1:1, and wherein the temperature of the secondary reaction product and the alcohol wash solution are in the range of from about 20° C. to about 100° C., wherein the wash time is in the range of from about 5 minutes to about 30 minutes;

f) isolating and removing impurities from the purified reaction product;

g) optionally repeating steps (e) and (f) for a number of times in the range of from about 1 to about 20;

h) optionally drying the purification reaction product.

The purified, moderately esterified polyol fatty acid polyesters of the present invention are believed to be particularly well suited for use in a variety of laundry, textile, food, lubricant, and cosmetic applications, containing less than about 5% polyol; less than about 5 ppm of residual reaction solvent; less than about 700 ppm of lower alkyl esters; less than about 2% of a soap and free fatty acid mixture; less than about 1% of ash; and having an acid value of less than about 2.

In one embodiment, the purified moderately esterified polyol polyester is a purified moderately esterified sucrose polyester containing less than about 2% sucrose; less than about 3 ppm of residual reaction solvent; less than about 600 ppm of lower alkyl esters; less than about 1% of a soap and free fatty acid mixture; less than about 0.5% of ash; and having an acid value of less than about 1.

### DETAILED DESCRIPTION OF THE INVENTION

The present invention encompasses esterification processes for the production of moderately esterified polyol fatty acid polyesters, in particular purified, moderately esterified polyol fatty acid polyesters. The present invention will now be described in detail with reference to specific embodiments.

A. Definitions

Various publications and patents are referenced throughout this disclosure. All references cited herein are hereby incorporated by reference. Unless otherwise indicated, all percentages and ratios are calculated by weight. All percentages and ratios are calculated based on the total dry composition unless otherwise indicated.

All component or composition levels are in reference to the active level of that component or composition, and are exclusive of impurities, for example, residual solvents or by-products, which may be present in commercially available sources.

Referred to herein are trade names for components including various ingredients utilized in the present invention. The inventors herein do not intend to be limited by materials under a certain trade name. Equivalent materials (e.g., those obtained from a different source under a different name or catalog number) to those referenced by trade name may be substituted and utilized in the compositions, kits, and methods herein.

As used herein, and unless otherwise indicated, the use of a numeric range to indicate the value of a given variable is not intended to be limited to just discrete points within that stated range. One of ordinary skill in the art will appreciate that the use of a numeric range to indicate the value of a variable is meant to include not just the values bounding the stated range, but also all values and sub-ranges contained therein. By way of example, consider variable X that is disclosed as having a value in the range of A to B. One of ordinary skill in the art will understand that variable X is meant to include all integer and non-integer values bounded by the stated range of A to B. Moreover, one of ordinary skill in the art will appreciate that the value of the variable also includes all combinations and/or permutations of sub-ranges bounded by the integer and non-integer values within and including A and B.

As used herein, the term “moderately esterified polyol polyester” is intended to include those esters of the polyol having a degree of esterification in excess of the degree of esterification of the polyol, but less than the degree of esterification of the highly esterified polyol fatty acid polyester. As used herein, the term “degree of esterification” refers to the molar average percentage of hydroxyl groups of a polyol composition that have been esterified.

In one embodiment of the present invention the polyol is sucrose having eight hydroxyl groups. The moderately esterified sucrose polyester preferably has a degree of esterification of from about 40% to about 80%. As used herein the degree of esterification calculation does not include non-esterified polyol compounds that may be present. As will be appreciated by the ordinarily skilled artisan, the degree of esterification of an esterified polyol polyester may also be expressed by the polyol polyester's I-bar (\overline{I}) value. As used herein, the term “I-bar (\overline{I}) ” is defined as the molar average number of hydroxyl groups of the polyol that have been esterified.

In one embodiment of the present invention the polyol is sucrose having eight hydroxyl groups. The moderately esterified sucrose polyester preferably has an I-bar value in the range of from about 3.2 to about 6.2. As used herein the I-bar calculation does not include non-esterified polyol compounds that may be present.

In the description of the invention various embodiments and/or individual features are disclosed. As will be apparent to the ordinarily skilled practitioner, all combinations of such embodiments and features are possible and can result in preferred executions of the present invention.

B. Processes for Synthesizing Purified, Moderately Esterified Polyol polyester Fatty Acid Compositions

In general, the processes for the preparation of purified, moderately esterified polyol fatty acid polyesters of the present invention comprise the steps of forming an initial reaction product from an initial reaction mixture; optionally neutralizing any remaining reaction catalyst; optionally forming a secondary reaction product to recover residual reaction components (e.g., solvent) via such processes as evaporation; purifying the reaction product to remove any impurities and/or unreacted components; and optionally drying the purified reaction product.

i) Initial Reaction Product

As used herein, “initial reaction product” refers to the product that is formed by reacting an initial reaction mixture in an inert atmosphere, for a period of time in the range of from about 30 minutes to about 6 hours, and at a temperature in the range of from about 80° C. to about 140° C.

The initial reaction mixture comprises a polyol portion, a highly esterified polyol fatty acid polyester, a solvent, and a catalyst. In one embodiment, the molar ratio of the catalyst to the highly esterified polyol fatty acid polyester is in the range of from about 0.01:1 to about 10:1, alternatively in the range of from about 0.1:1 to about 5:1, alternatively from about 0.25:1 to about 1:1, alternatively in the range of from about 0.4:1 to about 0.6:1. In one embodiment, the weight ratio of the solvent to the combined weight of the polyol portion, the highly esterified polyol ester fatty acid, and the catalyst is in the range of from about 0.01:1 to about 2:1, in another embodiment is in the range of from about 0.05:1 to about 1:1, alternatively, in the range of from about 0.1:1 to about 0.5:1. In one embodiment, the molar ratio of polyol and highly esterified polyol polyester should be chosen such that the final ratio of total fatty acid esters to total polyol backbones added is in the range of from about 3.2:1 to about 6.4:1.

In one embodiment of the present invention the polyol is sucrose and the highly esterified polyol fatty acid polyester is a sucrose polyester with a degree of esterification of about 95%.

As used herein, the term “polyol” is intended to include any aliphatic or aromatic compound containing at least two free hydroxyl groups. In practicing the processes disclosed herein, the selection of a suitable polyol is simply a matter of choice. For example, suitable polyols may be selected from the following classes: saturated and unsaturated straight and branched chain linear aliphatic; saturated and unsaturated cyclic aliphatic, including heterocyclic aliphatic; or mononuclear or polynuclear aromatics, including heterocyclic aromatics. Carbohydrates and glycols are exemplary polyols. Especially preferred glycols include glycerin. Monosaccharides suitable for use herein include, for example, mannose, galactose, arabinose, xylose, ribose, apiose, rhamnose, psicose, fructose, sorbose, tagitose, ribulose, xylulose, and erythrulose. Oligosaccharides suitable for use herein include, for example, maltose, kojibiose, nigerose, cellobiose, lactose, melibiose, gentiobiose, turanose, rutinose, trehalose, sucrose and raffinose. Polysaccharides suitable for use herein include, for example, amylose, glycogen, cellulose, chitin, inulin, agarose, zylans, mannan and galactans. Although sugar alcohols are not carbohydrates in a strict sense, the naturally occurring sugar alcohols are so closely related to the carbohydrates that they are also preferred for use herein. The sugar alcohols most widely distributed in nature and suitable for use herein are sorbitol, mannitol and galactitol.

Particular classes of materials suitable for use herein include monosaccharides, disaccharides and sugar alcohols. Other classes of materials include sugar ethers and alkoxylated polyols, such as polyethoxy glycerol.

In one embodiment of the present invention the polyol has on average at least four, alternatively at least about 5, alternatively about 8 hydroxyl groups capable of being esterified per polyol molecule.

Suitable esterified epoxide-extended polyols include esterified propoxylated glycerols prepared by reacting a propoxylated glycerol having from 2 to 100 oxypropylene units per glycerol with C₁₀-C₂₄ fatty acids or with C₁₀-C₂₄ fatty acid esters, as described in U.S. Pat. Nos. 4,983,329 and 5,175,323, respectively, and esterified propoxylated glycerols prepared by reacting an epoxide and a triglyceride with an aliphatic polyalcohol, as described in U.S. Pat. No. 5,304,665 or with an alkali metal or alkaline earth salt of an aliphatic alcohol, as described in U.S. Pat. No. 5,399,728. Other polyols include acylated propylene oxide-extended glycerols having a propoxylation index of above about 2, preferably in the range of from about 2 to about 8, more preferably about 5 or above, wherein the acyl groups are C₈-C₂₄, preferably C₁₄-C₁₈, compounds, as described in U.S. Pat. Nos. 5,603,978 and 5,641,534 and fatty acid-esterified propoxylated glycerols, as described in U.S. Pat. Nos. 5,589,217 and 5,597,605.

Other suitable esterified epoxide-extended polyols include esterified alkoxylated polysaccharides. In one embodiment, the esterified alkoxylated polysaccharides are esterified alkoxylated polysaccharides containing anhydromonosaccharide units, alternatively are esterified propoxylated polysaccharides containing anhydromonosaccharide units, as described in U.S. Pat. No. 5,273,772.

The polyol has a degree of esterification less than the degree of esterification of both the moderately esterified polyol polyester and the highly esterified polyol fatty acid polyester. The polyol portion may be a single type or class of polyol (e.g., sucrose) or may alternatively be a blend of two or more types or classes of polyols (e.g., a sugar alcohols, such as sorbitol; monosaccharides, such as fructose; and oligosaccharides, such as maltose).

As used herein, the term “highly esterified polyol fatty acid polyester” is intended to include those esters of a polyol with a degree of esterification in excess of the degree of esterification of both the polyol and the moderately esterified polyol polyester. In one embodiment of the invention the highly esterified polyol polyester has a degree of esterification of at least about 70%, while in yet another embodiment the highly esterified polyol polyester has a degree of esterification of at least about 90%, preferably at least about 95%.

A variety of processes are known in the art for the synthesis of highly esterified polyol fatty acid polyesters that are suitable for use in the processes of the present invention. Examples of such processes are detailed in U.S. Pat. No. 3,963,699, to Rizzi et al., disclosing a solvent-free transesterification process in which a mixture of a polyol (such as sucrose), a fatty acid lower alkyl ester (such as a fatty acid methyl ester), an alkali metal fatty acid soap, and a basic catalyst is heated to form a homogenous melt. Excess fatty acid lower alkyl ester is added to the melt to form the higher polyol fatty acid polyesters. The polyesters are then separated from the reaction mixture by any of the routinely used separation procedures; distillation or solvent extraction are preferred. Additional suitable processes include U.S. Pat. Nos. 4,517,360, to Volpenhein et al.; 5,422,131, to Elsen et al.; 5,648,483, to Granberg et al.; 5,767,257, to Schafermeyer et al., and 6,261,628, to Howie et al.

In one embodiment of the present invention, the highly esterified polyol fatty acid polyesters are sucrose fatty acid polyesters, having an average of at least 4 fatty acid groups per molecule. In another embodiment of the invention, the highly esterified polyol fatty acid polyester is sucrose fatty acid polyester having an average of at least 5 fatty acid groups per molecule, while in another embodiment the sucrose fatty acid polyesters have an average of from about 5 to about 8 fatty acid groups per molecule. In yet another embodiment, the polyol polyester is a sucrose polyester wherein at least about 75% of the sucrose polyester comprises octaester.

The fatty acid chains of the highly esterified polyol fatty acid polyesters may be branched, linear, saturated, unsaturated, hydrogenated, unhydrogenated, or mixtures thereof. The fatty acid chains of the fatty acid esters have from about 6 to about 30 total carbon atoms. As used herein, reference to a fatty acid compound having fatty acid chains of a particular length is intended to mean that a majority of the fatty acid chains, i.e., greater than 50 mol % of the fatty acid chains, have the stated length. In a more specific embodiment, the fatty acid compounds have greater than about 60 mol %, and more specifically greater than about 75 mol %, of fatty acid chains of the stated length. As used herein “fatty acid ester” is intended to include fatty acid esters in which the fatty acid chains have a total of from about 2 to about 28, typically from about 8 to about 22, carbon atoms. The fatty acid esters may be branched, unbranched, saturated, unsaturated, hydrogenated, unhydrogenated, or mixtures thereof.

In one embodiment of the present invention, the fatty acid chains of the polyester may be branched or linear and may be formed from fatty acid esters having fatty acid chains of from about 8 to about 26 total carbon atoms. In yet another embodiment, the fatty acid chains of the fatty acid ester have from about 16 to about 22 total carbon atoms.

Other suitable polyol fatty acid polyesters are esterified linked alkoxylated glycerins, including those comprising polyether glycol linking segments, as described in U.S. Pat. No. 5,374,446 and those comprising polycarboxylate linking segments, as described in U.S. Pat. Nos. 5,427,815 and 5,516,544.

Additional suitable polyol fatty acid polyesters include esterified epoxide-extended polyols of the general formula P(OH)_{A+C} (EPO)_{N} (FE)_{B} wherein P(OH) is a polyol, A is from 2 to about 8 primary hydroxyls, C is from about 0 to about 8 total secondary and tertiary hydroxyls, A+C is from about 3 to about 8, EPO is a C₃-C₆ epoxide, N is a minimum epoxylation index average number, FE is a fatty acid acyl moiety and B is an average number in the range of greater than 2 and no greater than A+C, as described in U.S. Pat. No. 4,861,613. The minimum epoxylation index average number has a value generally equal to or greater than A and is a number sufficient so that greater than 95% of the primary hydroxyls of the polyol are converted to secondary or tertiary hydroxyls. In one embodiment, the fatty acid acyl moiety has a C₇-C₂₃ alkyl chain.

The highly esterified polyol fatty acid polyester may be comprised of a single type or class of polyol polyester (e.g., sucrose) or may alternatively be a blend of two or more types or classes of polyol polyesters (e.g., a sugar alcohols, such as sorbitol; monosaccharides, such as fructose; and oligosaccharides, such as maltose). The polyol backbones of the highly esterified polyol fatty acid polyesters (e.g., sucrose in a highly esterified sucrose fatty acid polyester) may be the same backbone as the polyol, or may optionally be comprised of two or more different polyol backbones.

In one embodiment of the present invention the polyol is sucrose and the highly esterified polyol fatty acid polyester is predominantly (i.e., in excess of about 95%, preferably in excess of about 98%, more preferably in excess of about 99%) comprised of sucrose fatty acid polyester. In another embodiment the polyol is glucose and the highly esterified polyol fatty acid polyester is sucrose fatty acid polyester. In yet another embodiment, the polyol is sucrose and the highly esterified fatty acid polyester is comprised of sucrose fatty acid polyester and a highly esterified epoxide-extended polyol polyester.

Suitable basic compounds to be used as basic reaction catalysts include alkali metals such as sodium, lithium and potassium; alloys of two or more alkali metals such as sodium-lithium and sodium-potassium alloys; alkali metal hydrides, such as sodium, lithium and potassium hydride; alkali metal lower (C₁-C₄) alkyls such as butyl-lithium; and alkaline metal alkoxides of lower (C₁-C₄) alcohols, such as lithium methoxide, potassium t-butoxide, potassium methoxide, and/or sodium methoxide. Other suitable basic compounds include carbonates and bicarbonates of alkali metals or alkaline earth metals. Preferred classes of basic catalysts include potassium carbonate, sodium carbonate, barium carbonate, or mixtures of these compounds having particle sizes that are less than about 100 microns, preferably less than about 50 microns. These preferred catalysts could be used in admixture with the more conventional basic catalysts, described above. Potassium carbonate and/or potassium methoxide are also preferred catalysts. These catalysts are further disclosed in U.S. Pat. No. 4,517,360, to Volpenhein et al.

During the initial reaction phase it is preferable that the initial reaction mixture be as homogeneous as possible. A homogenous initial reaction mixture can be achieved by selection of appropriate reaction mixture ingredients that dissolve in the presence of the selected solvent. Examples of suitable solvents are selected from dimethyl sulfoxide, n-methyl formamide, dimethyl sulfate, formamide, dimethyl formamide, acetonitrile, acetone, and mixtures thereof. In one embodiment, dimethyl sulfoxide and dimethyl formamide are preferred solvents.

If the preferred degree of homogeneity is not readily achieved upon the admixing of the initial reaction mixture components, either by virtue of the ingredients or various other processing parameters selected, a sufficient amount of agitation may be applied during the initial reaction phase to form an approximately homogeneous mixture or emulsion. Agitation should be applied for a period of time necessary to maintain homogeneity throughout the duration of the initial reaction. Once agitation has been applied for a period of time necessary to assure homogeneity of the reactants throughout the reaction, further application of agitation may be continued, discontinued, or varied in force.

As used herein the term, “a sufficient amount of agitation” is defined as the level of agitation necessary to ensure that reaction components (e.g., the initial reaction mixture) do not separate into discrete phases for a period of time in excess of about 10 seconds, preferably in excess of about 20 seconds, more preferably in excess of about 30 seconds, more preferably in excess of about 45 seconds, most preferably in excess of about 60 seconds, following discontinuation of the agitation. In one embodiment, agitation is applied during the reaction for a period of time sufficient to ensure that the degree of esterification of the highly esterified polyol polyester fatty acid is reduced to below about 95%, preferably below about 90%, more preferably below about 80%.

In one embodiment of the present invention a heterogeneous initial reaction mixture comprises sucrose, a highly esterified sucrose fatty acid with a degree of esterification of about 95%, a potassium carbonate catalyst, and dimethyl sulfoxide (DMSO) as a solvent. Agitation is applied by use of a rotating impeller. The degree of agitation necessary to ensure a suitable degree of homogeneity throughout the reaction is quantified by a Weber Number in the range of from about 2000 to about 20,000, operating for a period of time in the range of from about 10 minutes to about 6 hours. In another embodiment the degree of agitation necessary to ensure suitable homogeneity is quantified by a Weber Number of about 10,000, applied for approximately 60 minutes. In yet another embodiment the agitation is quantified by a Weber Number of about 9,000 applied for the entire duration of a 120-minute reaction time.

As used herein, any device capable of inducing motion in the fluid reaction mixtures over a range of viscosities, thus effecting a dispersion of the components, is a suitable agitator for use in the processes of the present invention. Examples of suitable agitators include impellers, paddles, kneaders, helical rotors, single sigma blade, double sigma blades, screw-type agitators, ribbon agitators, and mixtures thereof.

As used herein, the “Weber Number” is a dimensionless number intended to provide a system independent measure of the agitation force applied to a reaction mixture. The Weber Number is defined by Equation 1.

$$\frac{\begin{matrix}( Density of the Continuous Phase ) \times \\ {( RPM of the Impellor )}^{2} \times \\ {( Diameter of the Impellor )}^{3}\end{matrix}}{\begin{matrix}Interfacial Tension between the Continuous \\ and Discontinuous Phases\end{matrix}} . \qquad Equation 1$$

ii) Catalyst Neutralization

Optionally, any catalyst remaining subsequent to the formation of the initial reaction product may be neutralized with an acid. Without being limited by theory, applicants have found that neutralization of the remaining catalyst reduces the risk of saponification and base catalyzed hydrolysis reactions during aqueous purification, both of which adversely impact the purity of the moderately esterified polyol fatty acid compositions.

To effectively neutralize any residual catalyst, a sufficient amount of an acid is added to the initial reaction product such that the molar ratio of the acid to total catalyst is in the range of from about 0.01:1 to about 1:1, preferably in the range of from about 0.1:1 to about 0.8:1, more preferably in the range of from about 0.6:1 to about 0.8:1. Examples of acids suitable for use in neutralizing any residual base catalyst include those acids selected from hydrochloric, phosphoric, chromic, iodic, benzoic, hydrofluoric, sulfuric, sulfurous, acetic, formic, nitric, and mixtures thereof.

iii) Secondary Reaction Product

Optionally, a secondary reaction product may be formed subsequent to the formation of the initial reaction product. Without being limited by theory, the primary purpose for forming a secondary reaction product is to recover various initial reaction mixture components, such as solvent, that are no longer required for the remaining purification processes. Additionally, removal of the solvent by formation of the secondary reaction product reduces the amount of solvent present in the final moderately esterified polyol fatty acid polyester compositions.

The secondary reaction product is formed by reacting the initial reaction product at a pressure in the range of from about 0.01 mmHg to about 760 mmHg, preferably in the range of from about 0.1 mmHg to about 20mmHg, more preferably in the range of from about 0 mmHg to about 10 mmHg, most preferably in the range of from about 0.1 mmHg to abut 5 mmHg, and for a period of time in the range of from about 30 minutes to about 4 hours.

In one embodiment of the present invention the desired reaction pressure dictates the temperature at which the secondary reaction product is formed. In another embodiment of the invention the desired reaction temperature dictates the reaction pressure to be employed. Preferably the secondary reaction product is formed at the temperature-pressure combination at which distillation of the solvent used in the initial reaction mixture occurs.

In yet another embodiment the solvent is dimethyl sulfoxide. Preferred temperature-pressure combinations for dimethyl sulfoxide are selected from about 0.0 mmHg and about −18° C., about 0.1 mmHg and about 4° C., about 0.5 mmHg and about 23° C., about 5 mmHg and about 58° C., about 10 mmHg and about 70° C., about 20 mmHg and about 85° C., and about 760 mmHg and about 189° C.

One of ordinary skill in the art will appreciate upon reading the disclosure herein that the temperatures disclosed in the preferred temperature-pressure combinations refer to the temperature of the reaction ingredients, not the temperature setting of the equipment used to heat the reaction components. The ordinarily skilled artisan will also appreciate that the temperatures are approximations based on the distillation temperatures of the pure solvent and may vary slightly depending on the degree of solvent purity.

In one embodiment of the present invention, the step of neutralizing any remaining catalyst is performed subsequent to the formation of the initial reaction product, but prior to the formation of a secondary reaction product. In another embodiment the secondary reaction product is formed subsequent to the formation of the initial reaction product, though prior to the neutralization of remaining catalyst. In yet another embodiment, the remaining catalyst is neutralized with an acid without the formation of a secondary reaction product. In yet another embodiment the secondary reaction product is formed, while the remaining catalyst is not neutralized.

iv) Purification

(a) Solvent Free Aqueous Purification Processes

The reaction products of the present invention may be purified by an aqueous purification process via application of a water washing solution. Without being limited by theory, it is believed that in order to obtain moderately esterified polyol polyester compositions with improved purity, the aqueous purification process should be free of any solvents that would adversely affect the finished product purity requirement for the composition's intended use. As any solvent added after formation of the initial reaction product must ultimately be removed via a purification process, it is particularly preferred that the aqueous purification process be a substantially solvent-free purification process, more preferably free of any measurable amount of solvent.

The water washing solution comprises about 100% water, which may optionally be distilled, purified, or de-ionized. The water washing solution may comprise from about 0.1% to about 5% of a salt and from about 95% to about 99.9% water. The water washing solution is applied over a period of time in the range of from about 2 minutes to about 30 minutes, preferably in the range of from about 5-10 minutes. The weight ratio of the water washing solution to the initial weight of the reaction product to be purified (e.g., initial reaction product; secondary reaction product; acid neutralized initial reaction product; or acid neutralized secondary reaction product) is in the range of from about 0.01:1 to about 1:1, alternatively in the range of from about 0.05:1 to about 0.5:1, alternatively in the range of from about 0.1:1 to about 0.3:1. The temperature of the water washing solution is in the range of from about 20° C. to about 100° C., and the temperature of the reaction product to be purified is in the range of from about 20° C. to about 100° C. In one embodiment, the temperature of the water washing solution is in the range of from about 20° C. to about 60° C. when the majority of the fatty acid esters are unsaturated, and in another embodiment, in the range of from about 30° C. to about 80° C. when the majority of the fatty acid esters are saturated.

Examples of salts suitable for optional use in the present invention include salts selected from calcium salts, magnesium salts, barium salts, sodium salts, potassium salts, cesium salts, and mixtures thereof. In one embodiment, salts are selected from lithium chloride, lithium bromide, lithium iodide, lithium sulfate, calcium chloride, calcium bromide, calcium iodide, calcium sulfate, magnesium chloride, magnesium bromide, magnesium iodide, magnesium sulfate, barium chloride, barium bromide, barium iodide, barium sulfate, sodium chloride, sodium bromide, sodium iodide, sodium sulfate, potassium chloride, potassium bromide, potassium iodide, potassium sulfate, cesium chloride, cesium bromide, cesium iodide, cesium sulfate, and mixtures thereof. In one embodiment, salts are selected from calcium chloride, calcium bromide, calcium iodide, calcium sulfate, and mixtures thereof.

Following application of the water washing solution, impurities, unreacted components, and reaction byproducts are collected and removed from the washed reaction product. The washed reaction product separates into two discrete layers. The bottom layer contains the impurities, solvent, reaction byproducts, and unreacted reaction components to be removed and discarded. The top layer contains the moderately esterified polyol fatty acid polyester. Optionally, the bottom layer may be collected and processed to recover and/or recycle any desired reaction ingredients and/or byproducts (e.g., polyol and solvent).

The purification process of washing the reaction product and separating and collecting the moderately esterified polyol polyester may optionally be performed one or more additional times, depending on product composition at the end of the purification cycle and the desired finished product purity specification. In one embodiment, the purification cycle is repeated in the range of from about 1 to about 20 times to achieve relatively high degrees of purification.

In one embodiment of the present invention the water washing purification steps are repeated in the range of from about 2 to about 10 times. The quantity of water washing solution to be used in each purification cycle is calculated based on the initial weight of the reaction product to be purified (i.e., the weight of the reaction product prior to the first purification cycle). In each cycle the weight ratio of the water washing solution to the initial weight of the washed reaction product to be purified (e.g. initial reaction product; secondary reaction product; acid neutralized initial reaction product; or acid neutralized secondary reaction product) is within the range of from about 0.01:1 to about 1:1, alternatively in the range of from about 0.05:1 to about 0.5:1, alternatively in the range of from about 0.1:1 to about 0.3:1.

The quantity of water washing solution utilized may be substantially the same for each purification cycle, or alternatively may vary from cycle to cycle. Additionally, the quantity of salt, if utilized in the water wash solution, may be substantially the same for each purification cycle, or alternatively may vary from cycle to cycle. Combinations of varying amounts of water and/or salt, if utilized, within the water washing solution of various purification cycles are also contemplated.

In one embodiment, the quantity of salt utilized in the water washing solutions of a purification cycle subsequent to the first purification cycle is less than the quantity of salt utilized in the previous purification cycle. In another embodiment, the quantity of salt utilized in the water washing solutions of a purification cycle subsequent to the first purification cycle is greater than the quantity of salt utilized in the previous purification cycle.

For each of the purification cycles the temperature of the water washing solution is in the range of from about 20° C. to about 100° C., and the temperature of the reaction product to be purified is in the range of from about 20° C. to about 100° C.

Optionally, the weight ratio of water washing solution to reaction product to be purified may be recalculated after each purification cycle, such that the weight ratio of the water washing solution to the weight of the reaction product to be purified in a given purification cycle is in the range of from about 0.01:1 to about 1:1, alternatively in the range of from about 0.05:1 to about 0.5:1, alternatively in the range of from about 0.1:1 to about 0.3:1.

(b) Alcohol Purification Processes

The reaction products of the present invention may optionally be purified by an alcohol purification process, via application of an alcohol washing solution. Without being limited by theory, it is believed that in order to obtain moderately esterified polyol polyester compositions with improved purity, the alcohol purification process should be free of any additional solvents that would adversely affect the finished product purity requirement for the composition's intended use. As any solvent added after formation of the initial reaction product must ultimately be removed via a purification process, it is preferred that the alcohol washing solution contain no additional ingredients that would not be substantially removed, preferably completely removed, by the alcohol wash process. Preferred embodiments of the present invention are those where the alcohol wash solution comprises no ingredients, other than perhaps impurities at a level that would not adversely impact finished product purity, beyond the alcohol.

The alcohol washing solution may include alcohols with a carbon chain length in the range of from about 2 atoms to about 5 atoms. The alcohol washing solution is applied over a period of time in the range of from about 2 minutes to about 30 minutes, alternatively in the range of from about 5-10 minutes. The weight ratio of the alcohol washing solution to the initial weight of the reaction product to be purified (e.g., initial reaction product; secondary reaction product; acid neutralized initial reaction product; or acid neutralized secondary reaction product) is in the range of from about 0.01:1 to about 1:1, alternatively in the range of from about 0.05:1 to about 0.5:1, alternatively in the range of from about 0.1:1 to about 0.3:1.

The temperature of the alcohol washing solution is in the range of from about 20° C. to about 100° C., and the temperature of the reaction product to be purified is in the range of from about 20° C. to about 100° C. Alternatively, the temperature of the alcohol washing solution is in the range of from about 20° C. to about 60° C. when the majority of the fatty acid esters are unsaturated, and in the range of from about 30° C. to about 80° C. when the majority of the fatty acid esters are saturated.

Examples of alcohols suitable for use in the present invention include ethanol, n-propanol, n-butanol, n-pentanol, branched and non-terminal forms of C₂-C₅ alcohols, and mixtures thereof. In one embodiment, alcohols are selected from ethanol, n-propanol, n-butanol, n-pentanol, and mixtures thereof.

Following application of the alcohol washing solution, impurities, unreacted components, and reaction byproducts are collected and removed from the washed reaction product. The washed reaction product separates into two discrete layers. The bottom layer contains the impurities, solvent, reaction byproducts, and unreacted reaction components to be removed and discarded. The top layer contains the moderately esterified polyol fatty acid polyester. Optionally, the bottom layer may be collected and processed to recover and/or recycle any desired reaction ingredients and/or byproducts (e.g., polyol and solvent).

Separation into the discrete phases may be accomplished by allowing the impurities and byproducts to gravity settle. Methods for the separation and isolation of impurities include centrifugation for a period of time in the range of from about 5 minutes to about 30 minutes at an applied force of from about 100 G to about 15000 G, alternatively in the range of from about 2000 G to about 10,000 G.

The purification cycle of washing the reaction product with alcohol and separating and collecting the moderately esterified polyol polyester may optionally be performed one or more additional times, depending on the product composition following the purification cycle and the desired degree of purity in the finished product. In one embodiment, the purification process is repeated in the range of from about 1 to about 20 times to achieve particularly high degrees of purification.

In one embodiment of the present invention the alcohol washing purification steps are repeated in the range of from about 2 to about 10 times. The quantity of alcohol washing solution to be used in each purification cycle is calculated based on the initial weight of the reaction product to be purified (i.e., the weight of the reaction product prior to the first purification cycle). In each cycle the weight ratio of the alcohol washing solution to the initial weight of the washed reaction product to be purified (e.g., initial reaction product; secondary reaction product; acid neutralized initial reaction product; or acid neutralized secondary reaction product) is within the range of from about 0.01:1 to about 1:1, alternatively in the range of from about 0.05:1 to about 0.5:1, alternatively in the range of from about 0.1:1 to about 0.3:1. The quantity of alcohol washing solution utilized may be substantially the same for each purification cycle, or alternatively may vary from cycle to cycle.

For each of the purification cycles the temperature of the alcohol washing solution is individually selected to be in the range of from about 20° C. to about 100° C., and the temperature of the reaction product to be purified is in the range of from about 20° C. to about 100° C.

Optionally, the weight ratio of alcohol washing solution to reaction product to be purified may be recalculated after each purification cycle, such that the weight ratio of the alcohol washing solution to the weight of the reaction product to be purified in a given purification cycle is in the range of from about 0.01:1 to about 1:1, alternatively in the range of from about 0.05:1 to about 0.5:1, alternatively in the range of from about 0.1:1 to about 0.3:1.

(c) Drying

Optionally, the purified moderately esterified polyol polyester fatty acid compositions of the present invention may be dried by a variety of water or alcohol removal techniques commonly known to those ordinarily skilled in the art. In one embodiment, the drying technique employed in the processes involves evaporation.

The purified, dried reaction product is formed by reacting the purified reaction product at a pressure in the range of from about 0.01 mmHg to about 760 mmHg, alternatively in the range of from about 0.1 mmHg to about 20 mmHg, alternatively in the range of from about 0.1 mmHg to about 10 mmHg, alternatively in the range of from about 0.1 mmHg to abut 5 mmH, and for a period of time in the range of from about 1 minutes to about 4 hours. The temperatures disclosed in the temperature-pressure combinations refer to the temperature of the reaction ingredients, not the temperature setting of the equipment used to heat the reaction components.

Subsequent to drying, the purified moderately esterified polyol polyester fatty acid compositions of the present invention that have been purified using water washing should have a Carl Fischer moisture content (as measured on a model MKA-510N Carl Fischer Moisture Titrator, produced by the Kyoto Electric manufacturing Company of Kyoto, Japan) of less than about 5%, preferably less than about 3%, more preferably less than about 1%, yet more preferably less than about 0.5%.

C. Composition of Purified, Moderately Esterified Polyol Fatty Acid Polyesters

The purified, moderately esterified polyol polyester fatty acid compositions of the present invention generally comprise a moderately esterified polyol polyester with a degree of esterification in the range of from about 40% to about 80%. Additionally, the purified, moderately esterified polyol polyester fatty acid compositions comprise less than about 5% polyol, preferably less than about 3.5% polyol, more preferably less than about 2% polyol, more preferably less than about 1.1% polyol; less than about 5% residual solvent, preferably less than 1000 ppm (parts per million) of residual solvent, preferably less than about 750 ppm of residual solvent, most preferably less than about 500 ppm of residual solvent; and less than about 700 ppm of lower alkyl esters, preferably less than about 650 ppm of lower alkyl esters, more preferably less than about 500 ppm of lower alkyl esters, more preferably less than about 200 ppm of lower alkyl esters, more preferably less than about 100 ppm of lower alkyl esters, most preferably less than about 50 ppm of lower alkyl esters. Moreover, the purified, moderately esterified polyol polyester compositions comprise less than about 5% of a soap and free fatty acid mixture, preferably less than about 4.5% of a soap and free fatty acid mixture, more preferably less than about 4% of a soap and free fatty acid mixture, more preferably less than about 3.5% of a soap and free fatty acid mixture, most preferably less than about 1% of a soap and free fatty acid mixture.

The purified, moderately esterified polyol polyesters also comprise less than about 3% ash, preferably less than about 2% ash, more preferably less than about 0.5% ash. As used herein, the term “ash” refers to sulfated ash. The amount of sulfated ash in the present invention is calculated by weighing 5 grams of a sample into a platinum dish. Then 5 mL of 10% Sulfuric acid (H₂SO₄) is added to the sample, and the mixture is heated until carbonized. The carbonized ash is then baked in a muffle furnace at 550° C. until ashed. An additional aliquot of 2-3 mL of 10% Sulfuric Acid is added, and the mixture is again heated until carbonized. Again the mixture is baked at 550° C. until ashed. This process is repeated until the ash maintains a constant weight. The percentage of sulfated ash is calculated by dividing the weight of the remaining ash by the sample weight.

Furthermore, the purified polyester compositions of the present invention have an acid value of less than about 4, preferably an acid value less than about 3, more preferably an acid value less than about 2, most preferably an acid value less than about 0.5.

Without being limited by theory, it is believed that residual levels of lower alkyl ester impurities may be attributed to those amounts that exist as an impurity within the highly esterified polyol polyester fatty acids prior to inclusion in the initial reaction mixture. Soap and free fatty acid mixtures are believed to be byproducts resulting from polyol degradation and catalyst neutralization reactions. Ash is also believed to be a byproduct of various degradation and purification processes within the synthesis of the purified, moderately esterified polyol polyester compositions.

The purified polyester compositions of the present invention are typically light to clear in color. As measured on a Lovibond Model PFX995 Colorimeter, (Manufactured by Tintometer Ltd., The Colour Laboratory of Salisbury, UK) the purified compositions of the present invention have a Lovibond Red Color measurement of less than about 20, preferably less than about 15, more preferably less that about 10, yet more preferably less than about 5.

### D. EXAMPLES

The following are non-limiting examples of moderately esterified polyol polyester and purified, moderately esterified polyol polyester compositions and methods of making the same, used in accordance with the present invention. The following examples are provided to illustrate the invention and are not intended to limit the spirit or scope thereof in any manner.

### Example 1

In the present example, an initial reaction mixture comprises 750 g (0.314 moles) of sucrose polyester, based on oleic fatty acids, with a degree of esterification of 96%; 11.5 g (0.0336 moles) of sucrose; 10 g (0.072 moles) of potassium carbonate; and 200 g of dimethyl formamide solvent. Prior to use in the initial reaction mixture the sucrose and catalyst are dried in a vacuum oven for 12 hours. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by supercritical fluid chromatography (SFC) and found to have the composition shown in Table 1A, wherein SEₓ indicates a Sucrose Ester with x esterified hydroxyl groups. Suitable super fluid chromatography analytical methods are described in U.S. Pat. No. 6,566,124, issued May 20, 2003 to Trout et al., entitled “Improved Processes for Synthesis and Purification of Nondigestible Fats.” The table below represents the weight percents of the various sucrose esters on a solvent-free basis.

TABLE 1A

| Soap | Sucrose | SE₁ | SE₂ | SE₃ | SE₄ | SE₅ | SE₆ | SE₇ | SE₈ |
|---|---|---|---|---|---|---|---|---|---|
| 0.4 | — | — | — | — | 0.5 | 3.5 | 20.0 | 39.9 | 35.7 |

The moderately esterified polyol polyester of Example 1 has a degree of esterification of about 87%.

### Example 2

In the present example, an initial reaction mixture comprises 750 g (0.314 moles) of sucrose polyester with a degree of esterification of 96%; 31.3 g (0.0916 moles) of sucrose; 10 g (0.072 moles) of potassium carbonate; and 300 g of dimethyl sulfoxide solvent. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by supercritical fluid chromatography (SFC) and found to have the composition shown in Table 2A.

TABLE 2A

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | — | — | 0.7 | 5.5 | 21.0 | 36.0 | 28.0 | 8.4 |

The initial reaction product has a degree of esterification of about 75%.

The initial reaction product is then purified with 109 g of deionized water. This water wash is carried out at 60° C. under mild agitation for 10 minutes. This purified reaction product is then centrifuged and the top product layer is decanted and the bottom water layer is discarded. The top product layer is then dried on a wiped film evaporator operating at 100° C. and 1 mmHg with a residence time of about 2 minutes. The purified, dried reaction product has a moisture content of about 0.2%. A sample of the purified, dried reaction product from the evaporator is retained and any water and/or other volatile impurities from the evaporator can be collected and recycled.

A sample of the purified, dried reaction product is analyzed by supercritical fluid chromatography (SFC) and found to have the composition shown in Table 2B.

TABLE 2B

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | — | — | 0.8 | 5.4 | 21.2 | 35.8 | 28.0 | 8.4 |

The dried purified reaction product has an acid value of about 0.5, a lower alkyl ester level of about 300 ppm, a DMSO level of about 50 ppm, and an ash level of about 0.2%.

### Example 3

In the present example, an initial reaction mixture comprises 750 g (0.314 moles) of sucrose polyester with a degree of esterification of 96%; 59.1 g (0.1727 moles) of sucrose; 10 g (0.072 moles) of potassium carbonate; and 300 g of dimethyl sulfoxide solvent. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by supercritical fluid chromatography (SFC) and found to have the composition shown in Table 3A.

TABLE 3A

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | 0.1 | 1.4 | 7.0 | 19.5 | 32.6 | 27.7 | 9.6 | 1.3 |

The initial reaction product has a degree of esterification of about 62%.

The initial reaction product is then purified with 170 g of deionized water. This water wash is carried out at 60° C. under mild agitation for 10 minutes. This purified reaction product is then centrifuged and the top product layer is decanted and the bottom water layer is discarded. The top product layer is then dried on a wiped film evaporator operating at 100° C. and 1 mmHg with a residence time of about 2 minutes. The purified, dried reaction product has a moisture content of about 0.3%. A sample of the purified, dried reaction product from the evaporator is retained and any water and/or other volatile impurities from the evaporator can be collected and recycled.

A sample of the purified, dried reaction product is analyzed by supercritical fluid chromatography (SFC) and found to have the composition shown in Table 3B.

TABLE 3B

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | 0.1 | 1.4 | 7.2 | 19.3 | 32.7 | 27.9 | 9.3 | 1.3 |

The dried purified reaction product has an acid value of about 0.7, a lower alkyl ester level of about 200 ppm, a DMSO level of about 50 ppm, and an ash level of about 0.1%.

### Example 4

In the present example, an initial reaction mixture comprises 750 g (0.314 moles) of sucrose polyester with a degree of esterification of 96%; 101 g (0.2944 moles) of sucrose; 10 g (0.072 moles) of potassium carbonate; and 400 g of dimethyl sulfoxide solvent. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by supercritical fluid chromatography (SFC) and found to have the composition shown in Table 4A.

TABLE 4A

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | 0.7 | 5.9 | 17.7 | 32.2 | 29.3 | 12.1 | 1.5 | — |

The initial reaction product has a degree of esterification of about 50%.

The initial reaction product is then purified with 150 g of deionized water. This water wash is carried out at 60° C. under mild agitation for 10 minutes. This purified reaction product is then centrifuged and the top product layer is decanted and the bottom water layer is discarded. The top product layer is then dried on a wiped film evaporator operating at 100° C. and 1 mmHg with a residence time of about 2 minutes. The purified, dried reaction product has a moisture content of about 0.2%. A sample of the purified, dried reaction product from the evaporator is retained and any water and/or other volatile impurities from the evaporator can be collected and recycled.

A sample of the purified, dried reaction product is analyzed by supercritical fluid chromatography (SFC) and found to have the composition shown in Table 4B.

TABLE 4B

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | 0.8 | 5.9 | 17.8 | 32.0 | 29.2 | 12.3 | 1.4 | — |

The dried purified reaction product has an acid value of about 0.4, a lower alkyl ester level of about 200 ppm, a DMSO level of about 40 ppm, and an ash level of about 0.1%.

### Example 5

In the present example, an initial reaction mixture comprises 750 g (0.314 moles) of sucrose polyester with a degree of esterification of 96%; 101 g (0.2944 moles) of sucrose; 10 g (0.072 moles) of potassium carbonate; and 400 g of dimethyl sulfoxide solvent. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by supercritical fluid chromatography (SFC) and found to have the composition shown in Table 5A.

TABLE 5A

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | 0.7 | 5.9 | 17.7 | 32.2 | 29.3 | 12.1 | 1.5 | — |

This represents a degree of esterification of about 50%.

The initial reaction product is then purified with 150 g of deionized water and 15 g of sodium chloride. This water wash is carried out at 60° C. under mild agitation for 10 minutes. This purified reaction product is then centrifuged and the top product layer is decanted and the bottom water layer is discarded. The top product layer is then dried on a wiped film evaporator operating at 100° C. and 1 mmHg with a residence time of about 2 minutes. The purified, dried reaction product has a moisture content of about 0.2%. A sample of the purified, dried reaction product from the evaporator is retained and any water and/or other volatile impurities from the evaporator can be collected and recycled.

A sample of the purified, dried reaction product is analyzed by supercritical fluid chromatography (SFC) and found to have the composition shown in Table 5B.

TABLE 5B

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | 0.8 | 5.9 | 17.8 | 32.0 | 29.2 | 12.3 | 1.4 | — |

The dried purified reaction product has an acid value of about 0.4, a lower alkyl ester level of about 200 ppm, a DMSO level of about 40 ppm, and an ash level of about 0.2%.

### Example 6

In the present example, an initial reaction mixture comprises 750 g (0.314 moles) of sucrose polyester with a degree of esterification of 96%; 101 g (0.2944 moles) of sucrose; 10 g (0.072 moles) of potassium carbonate; and 300 g of dimethyl sulfoxide solvent. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by supercritical fluid chromatography (SFC) and is found to have the composition shown in Table 6A.

TABLE 6A

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | 0.7 | 5.9 | 17.7 | 32.2 | 29.3 | 12.1 | 1.5 | — |

The initial reaction product has a degree of esterification of about 50%.

The initial reaction product is then purified with 150 g of methanol. This alcohol wash is carried out at 50° C. under mild agitation for 10 minutes. This purified reaction product is then centrifuged and the top product layer is decanted and the bottom alcohol layer is discarded. The top product layer is then dried on a wiped film evaporator operating at 100° C. and 1 mmHg with a residence time of about 2 minutes. The purified, dried reaction product has a methanol content of about 0.1%. A sample of the purified, dried reaction product from the evaporator is retained and any water and/or other volatile impurities from the evaporator can be collected and recycled.

A sample of the purified, dried reaction product is analyzed by supercritical fluid chromatography (SFC) and found to have the composition shown in Table 6B.

TABLE 6B

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | 0.8 | 5.9 | 17.8 | 32.0 | 29.2 | 12.3 | 1.4 | — |

The purified, dried reaction product has an acid value of about 0.5, a lower alkyl ester level of about 300 ppm, a DMSO level of about 50 ppm, and an ash level of about 0.2%.

### Example 7

In the present example, an initial reaction mixture comprises 750 g (0.314 moles) of sucrose polyester with a degree of esterification of 96%; 59.1 g (0.1727 moles) of sucrose; 10 g (0.072 moles) of potassium carbonate; and 300 g of dimethyl sulfoxide solvent. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by supercritical fluid chromatography (SFC) and found to have the composition shown in Table 7A.

TABLE 7A

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | 0.1 | 1.4 | 7.0 | 19.5 | 32.6 | 27.7 | 9.6 | 1.3 |

The initial reaction product has a degree of esterification of about 62%.

The initial reaction produce is then neutralized using 6.0 g of 36.5% hydrochloric acid in water.

A secondary reaction product is then formed by reacting the neutralized initial reaction product at 70° C. and 0.5 mmHg for 2 hours. Approximately 250 g of dimethyl sulfoxide is collected during this step. The secondary reaction product then weighs approximately 875 g.

The secondary reaction product is then purified with 100 g of deionized water. This water wash is carried out at 60° C. under mild agitation for 10 minutes. This purified secondary reaction product is then centrifuged and the top product layer is decanted and the bottom water layer is discarded. The top product layer is then dried on a wiped film evaporator operating at 100° C. and 1 mmHg with a residence time of 2 minutes. The purified, dried reaction product has a moisture content of about 0.2%. A sample of the purified, dried reaction product from the evaporator is retained and any water and/or other volatile impurities from the evaporator can be collected and recycled.

A sample of the dried purified reaction product is analyzed by supercritical fluid chromatography (SFC) and found to have the composition shown in Table 7B.

TABLE 7B

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | 0.1 | 1.4 | 7.2 | 19.3 | 32.7 | 27.9 | 9.3 | 1.3 |

The dried purified reaction product has an acid value of about 0.4, a lower alkyl ester level of about 350 ppm, a DMSO level of about 20 ppm, and an ash level of about 0.2%.

### Example 8

In the present example, an initial reaction mixture comprises 750 g (0.314 moles) of sucrose polyester with a degree of esterification of 96%; 59.1 g (0.1727 moles) of sucrose; 10 g (0.072 moles) of potassium carbonate; and 400 g of dimethyl sulfoxide solvent. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by supercritical fluid chromatography (SFC) and found to have the composition shown in Table 8A.

TABLE 8A

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | 0.1 | 1.4 | 7.0 | 19.5 | 32.4 | 27.9 | 9.7 | 1.2 |

The initial reaction product has a degree of esterification of about 62%.

The initial reaction product is then neutralized using 7.0 g of 36.5% hydrochloric acid in water.

A secondary reaction product is then formed by reacting the neutralized initial reaction product at 70° C. and 0.5 mmHg for 2 hours. Approximately 350 g of dimethyl sulfoxide is collected during this step. The secondary reaction product then weighs approximately 875 g.

The secondary reaction product is then purified with 100 g of methanol. This alcohol wash is carried out at 50° C. under mild agitation for 10 minutes. This purified secondary reaction product is then centrifuged and the top product layer is decanted and the bottom alcohol layer is discarded. The top product layer is then dried on a wiped film evaporator operating at 100° C. and 1 mmHg to with a residence time of 2 minutes. The purified, dried reaction product has a methanol content of about 0.1%. A sample of the purified, dried reaction product from the evaporator is retained and any water and/or other volatile impurities from the evaporator can be collected and recycled.

A sample of the purified, dried reaction product is analyzed by supercritical fluid chromatography (SFC) and found to have the composition shown in Table 8B.

TABLE 8B

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | <0.1% | 0.1 | 1.4 | 7.2 | 19.3 | 32.7 | 27.9 | 9.3 | 1.3 |

The purified, dried reaction product has an acid value of about 0.4, a lower alkyl ester level of about 350 ppm, a DMSO level of about 20 ppm, and an ash level of about 0.2.

Having now described several embodiments of the present invention it should be clear to those skilled in the art that the forgoing is illustrative only and not limiting, having been presented only by way of exemplification. Numerous other embodiments and modifications are contemplated as falling within the scope of the present invention as defined by the appended claims thereto.

1. A process for the preparation of a purified moderately esterified polyol fatty acid polyester composition comprising the steps of:
a) forming an initial reaction mixture, said initial reaction mixture comprising:
i) a polyol portion;
ii) a highly esterified polyol fatty acid polyester;
iii) a solvent; and,
iv) a catalyst,
wherein the molar ratio of said polyol portion to said highly esterified polyol polyester is chosen such that the final ratio of total fatty acid esters to total polyol backbones is in the range from about 3.2:1 to about 6.4:1; wherein the molar ratio of said catalyst to said highly esterified polyol polyester is in the range of from about 0.01:1 to about 10:1, and wherein the weight ratio of said solvent to the combined weight of said polyol portion, said highly esterified polyol polyester, and said catalyst is in the range of from about 0.01:1 to about 2:1; and
b) forming an initial reaction product by reacting said initial reaction mixture in an inert atmosphere, in the presence of a sufficient amount of agitation, for a period of time in the range of from about 30 minutes to about 6 hours, and at a temperature in the range of from about 80° C. to about 140° C.

2. The process of claim 1 wherein said polyol portion is sucrose, said highly esterified polyol polyester is a sucrose fatty acid polyester with a degree of esterification in excess of about 80%, wherein said catalyst is selected from sodium, lithium, potassium, sodium-lithium alloys, sodium-potassium alloys, sodium hydride, lithium hydride, potassium hydride, butyl-lithium, lithium methoxide, potassium t-butoxide, potassium methoxide, sodium methoxide, potassium carbonate, sodium carbonate, barium carbonate, and mixtures thereof, and wherein said solvent is selected from dimethyl sulfoxide, dimethyl formamide, acetonitrile, acetone, and mixtures thereof.

3. The process of claim 2, wherein in Step (b) agitation is applied at a Weber Number in the range of from about 5000 to about 15000, and for a period of time in the range of from about 30 minutes to about 6 hours.

4. The process of claim 3 wherein said highly esterified polyol polyester is highly esterified sucrose fatty acid polyester with a degree of esterification in excess of about 95%, said catalyst is selected from potassium carbonate, sodium carbonate, barium carbonate, and mixtures thereof; and wherein said solvent is dimethyl formamide.

5. A process for the preparation of a purified moderately esterified polyol fatty acid polyester composition comprising the steps of:
a) forming an initial reaction mixture, said initial reaction mixture comprising:
i) a polyol portion;
ii) a highly esterified polyol fatty acid polyester having a degree of esterification in excess of about 80%;
iii) a solvent; and,
iv) a catalyst,
wherein the molar ratio of said polyol portion to said highly esterified polyol polyester is chosen such that the final ratio of total fatty acid esters to total polyol backbones is in the range from about 3.2:1 to about 6.4:1; wherein the molar ratio of said catalyst to said highly esterified polyol polyester is in the range of from about 0.01:1 to about 10:1, and wherein the weight ratio of said solvent to the combined weight of said polyol portion, said highly esterified polyol polyester, and said catalyst is in the range of from about 0.01:1 to about 2:1;
b) forming an initial reaction product by reacting said initial reaction mixture in an inert atmosphere, in the presence of a sufficient amount of agitation, for a period of time in the range of from about 30 minutes to about 6 hours, and at a temperature in the range of from about 80° C. to about 140° C.;
c) adding an acid to the initial reaction product to neutralize any remaining catalyst, wherein the molar ratio of said acid to said catalyst is in the range of from about 0.01:1 to about 1:1;
d) forming a secondary reaction product by reacting said initial reaction product at a temperature-pressure combination at which distillation of said solvent occurs, wherein the pressure is in the range of from about 0.01 mmHg to about 760 mmHg, for a period of time in the range of from about 30 minutes to about 4 hours;
e) forming a purified reaction product by washing said secondary reaction product with a solvent free water washing solution, said solvent free water washing solution comprising:
(i) from about 0% to about 5% of a salt; and
(ii) from about 95% to about 100% water;
wherein the weight ratio of said water washing solution to said secondary reaction product is in the range of from about 0.01:1 to about 1:1, wherein the temperature of said secondary reaction product and said water wash solution are in the range of from about 20° C. to about 100° C., and wherein said wash time is in the range of from about 5 minutes to about 30 minutes;
f) isolating and removing impurities from said purified reaction product;
g) optionally repeating steps (e) and (f) for a number of times in the range of from about 1 to about 20; and,
h) optionally drying said purification reaction product.

6. The process of claim 5 wherein said polyol portion is sucrose and said highly esterified polyol polyester is highly esterified sucrose fatty acid polyester with a degree of esterification in excess of about 95%.

7. The process of claim 5 wherein said solvent is selected from dimethyl formamide, dimethyl sulfoxide, acetonitrile, acetone, and mixtures thereof.

8. The process of claim 7 wherein said solvent is dimethyl sulfoxide.

9. The process of claim 5 wherein said catalyst is selected from alkali metals, alloys of two or more alkali metals, alkali metal hydrides, alkali metal lower (C1-C4) alkyls, alkaline metal alkoxides of lower (C₁-C₄) alcohols, carbonates of alkali metals, carbonates of alkaline earth metals, bicarbonates of alkali metals, bicarbonates of alkaline earth metals, and mixtures thereof.

10. The process of claim 9 wherein said catalyst is potassium carbonate.

11. The process of claim 5, wherein in Step (b) agitation is applied at a Weber Number in the range of from about 5000 to about 15000, and for a period of time in the range of from about 30 minutes to about 6 hours.

12. The process of claim 5 wherein said polyol portion comprises a blend of at least two different polyols.

13. The process of claim 12 wherein said polyol portion comprises sucrose and at least one additional polyol other than sucrose.

14. The process of claim 5 wherein in Step (b) agitation is applied at a Weber Number in the range of from about 5000 to about 15000, and for a period of time in the range of from about 30 minutes to about 6 hours.

15. The process of claim 5 wherein said solvent is dimethyl sulfoxide, wherein said catalyst is potassium carbonate, wherein said highly esterified polyol polyester is highly esterified sucrose fatty acid polyester with a degree of esterification in excess of about 95%, and wherein in Step (f), isolation of said impurities occurs by centrifuging said purified reaction product for a period of time in the range of from about 5 minutes to about 30 minutes, at an applied force in the range of from about 100 G to about 20000 G.

16. The process of claim 5 wherein said highly esterified polyol polyester comprises at least two different highly esterified polyol polyesters.

17. The process of claim 16 wherein said highly esterified polyol polyester comprises a highly esterified sucrose polyester and at least one additional highly esterified polyol polyester.

18. The process of claim 17 wherein said polyol portion comprises sucrose and at least one additional polyol other than sucrose.

19. The process of Step 5 wherein in Step (f) isolation of said impurities occurs by a method selected from gravity settling, centrifugation, temperature reduction, and mixtures thereof.

20. The process of claim 5 wherein in Step (f), isolation of said impurities occurs by centrifuging said purified reaction product for a period of time in the range of from about 5 minutes to about 30 minutes, at an applied force in the range of from about 100 G to about 20000 G.

21. The process of claim 20 wherein isolation of said impurities occurs by centrifuging said purified reaction product for a period of time in the range of from about 5 minutes to about 15 minutes, at an applied force in the range of from about 2500 G to about 10000 G.

22. The process of claim 20 wherein said purified reaction product comprises polyol fatty acid polyesters having in excess of about 50% unsaturated fatty acid esters, and wherein in Step (f) isolation of said impurities occurs by centrifuging said purified reaction product for a period of time in the range of from about 5 minutes to about 30 minutes, at an applied force in the range of from about 100 G to about 20000 G.

23. The process of claim 20 wherein in step (g), the steps (e) and (f) are repeated for a number of times in the range of from about 1 to about 20 times.

24. A process for the preparation of a purified moderately esterified sucrose fatty acid polyester composition comprising the steps of:
a) forming an initial reaction mixture, said initial reaction mixture comprising:
i) a sucrose polyol portion;
ii) a highly esterified sucrose fatty acid polyester having a degree of esterification in excess of 80%;
iii) a solvent selected from dimethyl formamide, dimethyl sulfoxide, acetonitrile, acetone, and mixtures thereof; and,
iv) a catalyst,
wherein the molar ratio of said polyol portion to said highly esterified polyol polyester is chosen such that the final ratio of total fatty acid esters to total polyol backbones is in the range from about 3.2:1 to about 6.4:1; wherein the molar ratio of said catalyst to said highly esterified polyol polyester is in the range of from about 0.01:1 to about 10:1, and wherein the weight ratio of said solvent to the combined weight of said polyol portion, said highly esterified polyol polyester, and said catalyst is in the range of from about 0.01:1 to about 2:1;
b) forming an initial reaction product by reacting said initial reaction mixture in an inert atmosphere selected from nitrogen, argon, helium, and combinations thereof, in the presence of a sufficient amount of agitation, for a period of time in the range of from about 30 minutes to about 6 hours, and at a temperature in the range of from about 80° C. to about 140° C.;
c) adding an acid to the initial reaction product to neutralize any remaining catalyst, wherein the molar ratio of said acid to said catalyst is in the range of from about 0.01:1 to about 1:1;
d) forming a secondary reaction product by reacting said initial reaction product at a temperature-pressure combination at which distillation of said solvent occurs, wherein the pressure is in the range of from about 0.01 mmHg to about 760 mmHg, for a period of time in the range of from about 30 minutes to about 4 hours;
e) forming a purified reaction product by washing said secondary reaction product with an alcohol washing solution, wherein said alcohol washing solution comprises an alcohol selected from alcohols with a carbon chain length in the range of from about 2 atoms to about 5 atoms, wherein the weight ratio of said alcohol washing solution to said secondary reaction product is in the range of from about 0.01:1 to about 1:1, and wherein the temperature of said secondary reaction product and said alcohol wash solution are in the range of from about 20° C. to about 100° C., wherein said wash time is in the range of from about 5 minutes to about 30 minutes;
f) isolating and removing impurities from said purified reaction product;
g) optionally repeating steps (e) and (f) for a number of times in the range of from about 1 to about 20;
h) optionally drying said purified reaction product.
